(19) United States
(12) Patent Application Publication
(10) Pub. No.: US 2025/0085230 A1
(43) Pub. Date: Mar. 13, 2025

(54) Gemstone Testing Apparatus

(71) Applicant: Jubilee Diamond Instrument (S) Pte. Ltd., Singapore (SG)

(72) Inventor: Kui Lim Tam, Singapore (SG)

(73) Assignee: Jubilee Diamond Instrument (S) Pte. Ltd., Singapore (SG)

(21) Appl. No.: 18/955,325

(22) Filed: Nov. 21, 2024

Related U.S. Application Data
(63) Continuation-in-part of application No. 18/306,892, filed on Apr. 25, 2023, now Pat. No. 12,174,126.
Application No. 18/306,892 is a continuation of application No. 17/302,980, filed on May 18, 2021, now Pat. No. 11,668,654.
Application No. 17/302,980 is a continuation of application No. 16/479,823, filed on Jul. 22, 2019, now abandoned.

(51) Int. Cl. G01N 21/87; G01N 21/33

(52) U.S. Cl. CPC G01N 21/87; G01N 21/33; G01N 33/389; G01N 2201/0221

(57) ABSTRACT

The application provides a gemstone testing apparatus for testing a specimen. The gemstone testing apparatus includes a handheld casing, a plurality of light sources, a test probe, a photodetector, a processor unit, and a display unit. The test probe is placed at one end of the handheld casing. A first end of the test probe is placed outside the handheld casing. The plurality of light sources is provided for emitting light rays towards an area that is in the vicinity of the first end. The first end is adapted for receiving light rays from the specimen and for transmitting the light rays to a second end of the test probe. The photodetector is arranged to measure an intensity of the light rays from the second end. The processor unit is provided for determining a material of the specimen in accordance to a measurement of the intensity of the light rays.

### CROSS-REFERENCE TO RELATED APPLICATIONS

This application is a Continuation-In-Part of U.S. application Ser. No. 18/306,892, titled “Gemstone Testing Apparatus” by Kui Lim Tam, filed Apr. 25, 2023, which is a Continuation of U.S. application Ser. No. 17/302,980, titled “Gemstone Testing Apparatus” by Kui Lim Tam, filed May 18, 2021, issued as U.S. Pat. No. 11,668,654 on Jun. 6, 2023, which is a Continuation of U.S. application Ser. No. 16/479,823, titled “Gemstone Testing Apparatus” by Kui Lim Tam, filed Jul. 22, 2019, which is a Continuation of U.S. application Ser. No. 16/479,823, titled “Gemstone Testing Apparatus” by Kui Lim Tam, filed on Jul. 22, 2019. U.S. application Ser. No. 16/479,823 is a National Stage Entry of International Application No. PCT/IB2017/058093, titled “Gemstone Testing Apparatus” by Jubilee Diamond Instrument(s) PTE. LTD., filed on Dec. 19, 2017.

This application incorporates the entire contents of the foregoing applications herein by reference.

This application contains related subject matter by a common inventor with
U.S. Ser. No. 15/751,529, titled “DIAMOND IDENTIFICATION APPARATUS,” filed by Kui Lim Tam on Feb. 9, 2018, issued as U.S. Pat. No. 10,161,878 on Dec. 25, 2018;
U.S. Ser. No. 16/496,721, titled “UV REFLECTION TESTER,” filed by Kui Lim Tam on May 31, 2018, issued as U.S. Pat. No. 10,989,667 on Apr. 27, 2021;
U.S. Ser. No. 16/128,610, titled “PRECIOUS STONE TESTING DEVICE,” filed by Kui Lim Tam on Sep. 12, 2018, issued as U.S. Pat. No. 10,228,830 on Mar. 12, 2019;
U.S. Ser. No. 16/176,059, titled “DIAMOND IDENTIFICATION APPARATUS,” filed by Kui Lim Tam on Oct. 31, 2018, issued as U.S. Pat. No. 10,247,677 on Apr. 2, 2019; and,
U.S. patent application Ser. No. 16/820,078, titled “GEMSTONE TESTING APPARATUS,” filed by Kui Lim Tam on Mar. 16, 2020, issued as U.S. Pat. No. 10,837,915 on Nov. 17, 2020.

This application incorporates the entire contents of the foregoing applications herein by reference.

### TECHNICAL FIELD

This application relates to an apparatus for testing gemstones, namely diamond and moissanite.

### BACKGROUND

A diamond includes a native crystalline carbon that is very hard. The diamond can have color or be colorless. When the diamond is transparent and free from flaws, it is highly valued as a jewelry. It is often used industrially as an abrasive.

Moissanite refers to a silicon carbide mineral and to its various crystalline polymorphs. The silicon carbide mineral can be found in nature, although this is rare. It can also be synthesized in the laboratory.

Synthetic moissanite, which is colorless or near-colorless, resembles diamond in many aspects, such as visual characteristics, hardness, and thermal conductivity among other physical properties. Therefore, synthetic moissanite is widely used as a diamond simulant in today's jewelry market.

A gemstone tester is often considered as a convenient tool for identifying gemstones, such as diamond, moissanite, and other precious stones. The gemstone tester can include a testing probe for determining thermal conductivity or electrical conductivity of the gemstone in order to classify the gemstone according to the thermal and electrical conductivity.

### SUMMARY

US20160363576A1 discloses a multi-functional precious stone testing apparatus that includes portable housing, a testing unit, and an indication unit. The portable housing includes a hand-held casing and a probe casing. The probe casing extends from a front end of the hand-held casing. The testing unit includes a conductive probe. The conductive probe has a testing end portion that extends out of a tip end of the probe casing. The indication unit includes a LED light unit. The LED light unit is placed in the hand-held casing. The LED light unit is also positioned away from the tip end of the probe casing. Functionally, the conductive probe is intended for contacting a testing object to determine a conductivity of the testing object. The LED light unit, which is received in the hand-held casing, is used for illuminating the testing end portion of the conductive probe during testing. The LED light unit, which is also positioned away from the tip end of the probe casing, also acts to prevent heat, which is generated from the LED light unit, from being transmitted toward the conductive probe. The heat can affect an accuracy of measurement for the conductivity of the testing object.

U.S. Pat. No. 6,043,742A discloses an apparatus for detecting man-made gem-stones using an alternating current conducted through a sample gemstone. The apparatus includes a hand-held housing in which is disposed electronic circuitry, a probe which extends from the housing, and a transmitting stimulus electrode in the form of a body-contact touchpad. The electronic circuitry includes a filter for eliminating non-transmitted signals sensed by the probe. In use, the operator probes the gemstone by touching the conductive probe to the gemstone in an attempt to sense signals conducted through the gemstone. The electronic circuitry is used for producing an alternating current signal, preferably in sine wave form, for delivery to the touchpad. The alternating current signal is transmitted through the operator of the apparatus into the sample gemstone. An alarm is activated upon the detection of the conducted transmitted signal, indicating that the gemstone is man-made.

It is an objective of the application to provide an improved gemstone testing apparatus.

The application provides an improved gemstone testing apparatus for testing a gemstone specimen in order to identify the material of the specimen. Examples of the gemstone specimen are diamonds and moissanites.

A thermal conductivity test is often used to separate diamond and moissanite from all other gemstones. Thereafter, the gemstone testing apparatus can be used to differentiate between diamond and moissanite.

The gemstone testing apparatus includes a handheld casing, a plurality of light sources, a test probe, a photodetector, a processor unit, and a display unit.

The light sources, a part of the test probe, the photodetector, and the processor are often placed inside the handheld casing. The display unit is often placed on the outer surface of the hand-held casing.

The handheld casing acts to contain and protect inner parts of the gemstone testing apparatus. The shape of the handheld casing is designed for allowing a user to easily hold or carry the gemstone testing apparatus. The handheld casing can include grip indentations on an outer surface of the gemstone testing apparatus, thereby allowing the user to maintain a firm hold of the gemstone testing apparatus. The handheld casing is often made of plastic material to reduce weight and cost.

A first end of the test probe is placed outside the handheld casing. A second end of the test probe is often placed inside the handheld casing. In other words, the test probe protrudes from one part of the handheld casing.

The plurality of light sources is placed on at least two sides of the test probe. The multiple light sources are provided for emitting ultraviolet (UV) light rays with a predetermined wavelength. The light sources are inclined at a predetermined angle in order to direct the light rays towards an area that is in the vicinity of the first end of the test probe. UV light rays normally refer to an electromagnetic radiation with a wavelength from about 10 nm to about 400 nm, although the workable range can be narrower.

The specimen is intended to be placed at this area for receiving the light rays. If the specimen is a diamond, it would reflect the light rays. If the specimen is a moissanite, it would absorb the light rays. In other words, the moissanite would not reflect the light rays.

In use, the test probe is placed near to the specimen. The first end of the test probe is adapted for receiving light rays from the specimen, which is illuminated by light rays from the multiple light sources. The test probe then transmits these light rays to the second end of the test probe.

The test probe is often provided with a light guide, such as a tube with a reflective inner surface. One end of the test probe is intended to receive light rays. An inner surface of the test probe then reflects and directs the light rays to an-other end of the test probe.

The photodetector is often placed near to the second end of the test probe. The photodetector is arranged to detect light rays from the light sources with the predetermined wavelength. These light rays travelled from the specimen, to the first end, to the second end of the test probe, and to the photodetector.

The photodetector is also arranged to measure a light intensity of these light rays.

The processor unit of an electronic testing unit is electrically connected to the plurality of light sources and to the photodetector. The processor unit is provided for determining a material of the gemstone in accordance to a measurement of the light intensity of the light rays. In other words, the processor unit determines whether the specimen includes a diamond or a moissanite.

If the processor unit determines that the specimen reflects light rays, then the processor unit considers the specimen is a diamond. On the other hand, if the processor unit determines that the specimen does not reflect light rays, then the processor unit considers the specimen is a moissanite.

The display unit is electrically connected to the processor unit. The display unit is used for receiving a data regarding the determination of the material of the gemstone specimen from the processor unit. The display unit then shows or displays this data.

The plurality of light sources provides benefits.

The arrangement of the multiple light sources allows a table of the specimen to receive light rays while the test probe is placed at different parts of the table of the specimen, even at an edge of the table. The table refers to a flat facet on the top of a gemstone specimen. This flat facet is often the largest facet of a cut specimen.

In practice, the size of the test probe is often smaller than the size of the table of the specimen. A user may place the test probe at different parts of the table of the specimen.

When the test probe is placed substantially near or at the center location of the table, the table of the specimen would receive light rays emitted from all multiple light sources.

When the test probe is not placed substantially near the center location of the table of the specimen, such as at the edge of the table, the table of the specimen would still receive light rays emitted from one or more of the multiple light sources.

In short, the multiple light sources allow the specimen to receive sufficient light rays for testing the specimen, even when the test probe is placed at different parts of the table of the gemstone. The user is not restricted to place the test probe at the center of the table in order to obtain an accurate gemstone test result.

This is different from other gemstone testers with a test probe and with just one single light source being placed at one side of the test probe.

When the test probe is placed near or at a center location of a table of a specimen, the table of the specimen would receive light rays from the single light source.

When the test probe is placed at an edge of the table of the specimen, only a side facet of the specimen may receive light rays from the single light source. In other words, no light rays or little light rays are directed onto the table of the specimen.

The table of the specimen may then not receive enough light rays for testing the specimen. This then degrades or affects the testing to the specimen.

The gemstone testing apparatus can have different aspects.

In one implementation, the plurality of light sources of the gemstone testing apparatus includes two light sources, although it can also include three or more light sources.

In a further implementation, the plurality of light sources is arranged around the test probe in a symmetric manner. In other words, the multiple light sources serve as similar parts that face each other or around a longitudinal axis of the test probe. In one example, two light sources are placed at two opposing sides of the test probe.

Each of the light sources is often inclined at a predetermined angle with respect to the longitudinal axis of the test probe.

The gemstone testing apparatus often includes a pressure switch and a pressure transmitting means.

In use, the test probe is brought in contact with a gemstone specimen, and it is pressed against a table or a surface of the gemstone specimen. The pressing acts for transferring a force from the gemstone specimen to the test probe. The pressure transmitting means acts to transfer the force from the test probe to the pressure switch. Upon receiving the force from the pressure transmitting means, the pressure switch then transmits a signal to activate or power up the processor unit of the gemstone testing apparatus. The activated processor unit thereafter provides electrical power to the multiple light sources for illuminating the gemstone specimen for testing the specimen.

The pressure switch together with the pressure transmitting means allows the multiple light sources to be powered up only when the gemstone testing apparatus is activated by the test probe pressing against the gemstone specimen. This thereby saves power. This is especially important when the gemstone testing apparatus is powered by a battery.

As an example, the pressure transmitting means includes an actuator member that includes a rod-like member. The rod-like member can also operate with a spring member. In use, when the actuator member is moved by the test probe, the actuator member shifts towards the pressure switch, wherein the actuator member pushes an on/off button of the pressure switch for activating the processor unit of the gemstone testing apparatus.

In one implementation, the pressure switch is provided in the form of a micro-switch. The micro-switch is normally in an open position. Upon receiving a force from the pressure transmitting means, the micro-switch changes to a closed position. The micro-switch provides a switch position signal to the processor unit for activating or powering up the processor unit in order to enable the gemstone testing apparatus to test the gemstone specimen.

In one aspect of the application, the multiple light sources of the gemstone testing apparatus emit light rays with a fixed wavelength that is between about 315 nm and about 400 nm while the photodetector is configured to detect light rays with this fixed wavelength. In other words, the photodetector with a peak detection sensitivity that is suitable for detecting light rays with this fixed wavelength.

Alternatively, the light rays can also have different wavelengths that are between about 315 nm and about 400 nm. The photodetector is then configured to detect light rays with these different wavelengths.

In one specific implementation, the multiple light sources emit light rays with a fixed wavelength of about 365 nm. The photodetector is configured to detect light rays with this fixed wavelength of about 365 nm.

In a different implementation, the plurality of light sources is replaced with a ring light. The ring light is arranged to surround the test probe. The ring light is used for emitting light rays from different sides of the test probe, wherein the light rays are directed towards the gemstone specimen.

The gemstone testing apparatus often includes an external cap that is intended for attaching to the handheld casing in order to cover and to protect the test probe from being damaged.

The external cap can further include a gemstone test reference tablet. The gemstone test reference tablet is capable of reflecting light rays from the multiple light sources. In use, a user uses the gemstone test reference tablet to check functions of the gemstone testing apparatus.

The gemstone testing apparatus often includes a power source unit for supplying electrical power to parts of the gemstone testing apparatus, such as the multiple light sources, the photodetector, the electronic testing unit, and the display unit.

The gemstone testing apparatus can provide gemstone test results to the user in different ways.

In one implementation, the display unit of the gemstone testing apparatus includes a plurality of indicator lights for providing visual indications of the gemstone test results. In other words, the display unit can include indicator lights or a display screen for emitting light rays to visually display data regarding the gemstone test results.

In another implementation, the gemstone testing apparatus further includes a buzzer or an audio speaker for generating an audio indication of the gemstone test result. An example of the audio indication includes a continuous or an intermittent beeping sound.

In another aspect of the application, the test probe includes a hollow light guide with a reflective inner surface. The reflective inner surface serves to reflect and direct light rays from one end to another end of the light guide.

In one specific implementation, the hollow light guide includes a metal tube.

The application also provides a method for differentiating between a diamond and a moissanite.

The method includes a step of a user pressing a test probe of the gemstone testing apparatus against a table of a gemstone specimen. A force is then transmitted from the gemstone to the test probe and to a pressure switch of the gemstone testing apparatus.

After this, a plurality of light sources of the gemstone testing apparatus is activated for illuminating the gemstone specimen with light rays. The table of the gemstone specimen receives the light rays from at least one of the multiple light sources.

If the gemstone specimen is a moissanite, the light rays are then absorbed. In other words, essentially no light rays are reflected from the moissanite. If the gemstone specimen is a diamond, the light rays are reflected to the test probe. An intensity of the light rays being reflected from the gemstone specimen is measured afterward. A material of the gemstone is later determined in accordance to the measured light intensity.

The method can include a further step of providing an indication of the material of the gemstone specimen to a user.

In one implementation, said step of providing the indication of the material of the gemstone specimen includes a plurality of light indicators providing a visual indication of the determined material of the gemstone specimen.

In another implementation, said step of providing the indication of the material of the gemstone specimen includes a buzzer or speaker generating an audio indication of the determined material of the gemstone specimen.

### BRIEF DESCRIPTION OF THE DRAWINGS

The subject matter of the application is described in greater detail in the accompanying Figures, in which,

FIG. 1 illustrates a perspective view of an improved gemstone testing apparatus,

FIG. 2 illustrates a rear view of the gemstone testing apparatus of FIG. 1;

FIG. 3 illustrates a partial cross-sectional view of a head portion of the gemstone testing apparatus of FIG. 1,

FIG. 4 illustrates an electronic block diagram of the gemstone testing apparatus of FIG. 1,

FIG. 5 illustrates a partial cross-sectional view of the head portion of the gemstone testing apparatus of FIG. 1, wherein a metal tube of the gemstone testing apparatus is placed at a center portion of a table of a specimen,

FIG. 6 illustrates a partial cross-sectional view of the head portion of the gemstone testing apparatus of FIG. 1, wherein the metal tube is placed at an edge of the table of specimen,

FIG. 7 illustrates a partial cross-sectional view of a head portion of another gemstone tester with a single light source, wherein a test probe of the gemstone tester is placed at a center portion of the table of the specimen,

FIG. 8 illustrates a partial cross-sectional view of the 16 head portion of the gemstone testing apparatus of FIG. 7, wherein the test probe is placed at an edge of the table of specimen,

FIG. 9 illustrates a top view of an external cap with a gemstone test reference tablet for the gemstone testing apparatus of FIG. 1,

FIG. 10 illustrates a flow chart of steps of a method of operating the gemstone testing apparatus of FIG. 1, and

FIG. 11 illustrates a partial side view of the gemstone test reference tablet of FIG. 9.

FIG. 12 illustrates an electronic block diagram of an exemplary gemstone testing apparatus capable of conducting either or both electrical conductivity testing and ultraviolet light-based gemstone evaluation.

FIG. 13 illustrates a flowchart depicting a n exemplary method for evaluating a gemstone specimen using both ultraviolet light intensity measurements and electrical conductivity testing.

FIG. 14 illustrates an internal configuration of the gemstone testing apparatus as described with reference to FIG. 12.

FIG. 15a and FIG. 15b illustrate a flow chart depicting an exemplary method to evaluate a gemstone specimen.

FIG. 16a and FIG. 16b illustrate a flowchart representing an exemplary method for operating the gemstone testing apparatus as described with reference to FIG. 12.

Like reference symbols in the various drawings indicate like elements.

### DETAILED DESCRIPTION OF ILLUSTRATIVE EMBODIMENTS

In the following description, details are provided to describe the embodiments of the specification. It shall be apparent to one skilled in the art, however, that the embodiments may be practiced without such details.

Some parts of the embodiments have similar parts. The similar parts may have the same names or similar part numbers with an alphabet symbol or prime symbol. The description of one part applies by reference to another similar part, where appropriate, thereby reducing repetition of text without limiting the disclosure.

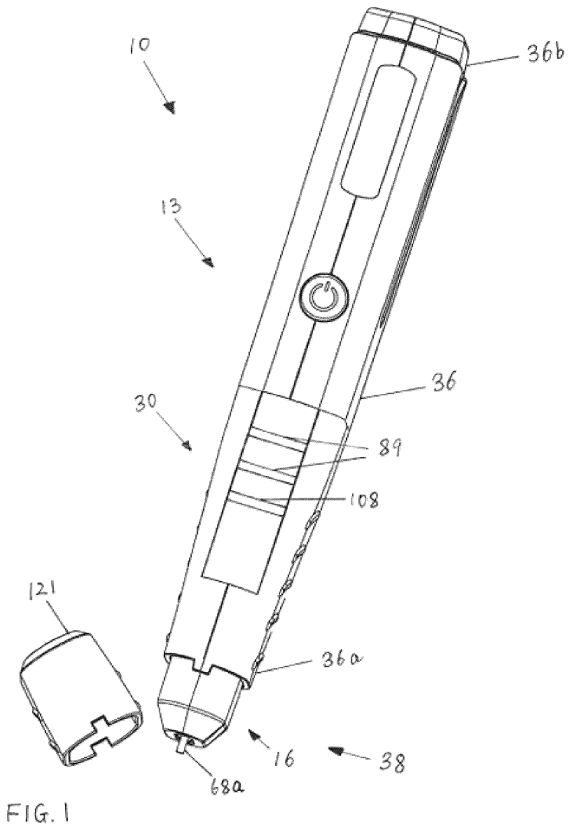

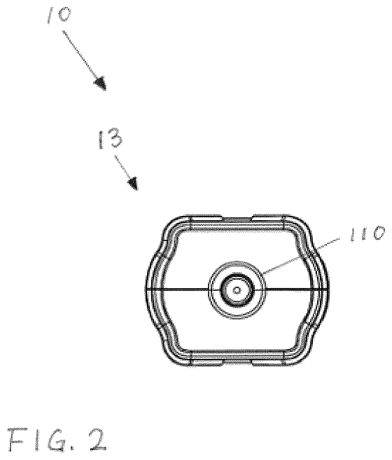

FIGS. 1 to 3 show an improved gemstone testing apparatus 10 to differentiate between diamond and moissanite.

In use, a gemstone specimen is often subjected to a thermal conductivity test. If the thermal conductivity test indicates that the specimen can be a moissanite or a diamond, the gem-stone testing apparatus 10 is then used to determine whether the specimen is a moissanite or a diamond.

The gemstone testing apparatus 10 includes an elongated handheld casing 13, a test probe 16 with a light module 19 together with a photodetector 21, a pressure switch 25, an electronic testing unit 28, a display unit 30 together with a buzzer 92, and a power source unit 33. The electronic testing unit 28 is also called a testing electronic circuit. The photodetector 21 is also called an ultraviolet (UV) sensor. The test probe 16 is also called a detector probe. The handheld casing 13 is also called an apparatus body. The power source unit 33 is also called a power source for short.

A part of the test probe 16, the light module 19, the photodetector 21, the pressure switch 25, the electronic testing unit 28, a part of the power source unit 33, and the buzzer 92 are placed inside the elongated handheld casing 13. The display unit 30 is placed on an outer surface of the elongated handheld casing 13. The electronic testing unit 28 is electrically connected to the power source unit 33, to the light mod-ule 19, to the photodetector 21, to the pressure switch 25, to the display unit 30, and to the buzzer 92. The electronic testing unit 28 is soldered on a printed circuit board (PCB).

The handheld casing 13 includes an elongated hollow body portion 36, a head portion 38, and a spring support unit 40.

The elongated hollow body portion 36 essentially has a shape of a cylinder. The elongated hollow body portion 36 has a first end 36a and a second end 36b, which is positioned opposite to the first end 36a. The head portion 38 is positioned next to the first end 36a of the elongated hollow body portion 36. A longitudinal axis of the elongated hollow body portion 36 is aligned with a longitudinal axis of the head portion 38. The spring support unit 40 is placed inside the elongated hollow body portion 36 and is attached to the head portion 38.

The head portion 38 includes a hollow conical member 42 with an actuator member 44 and a support member 47. The actuator member 44 is integrally connected to the hollow conical member 42. The hollow conical member 42 is placed next to the first to end 36a of the hollow body portion 36 of the handheld casing 13. The actuator member 44 and the support member 47 are placed inside the first end 36a of the hollow body portion 36. The actuator member 44 is movably connected to the support member 47. The support member 47 is fixed to the hollow body portion 36 of the handheld casing 13.

The spring support unit 40 includes a plurality of coil torsion springs 50. Parts of the actuator member 44 are inserted into the coil torsion springs 50. The coil torsion springs 50 are adapted for pushing the support member 47 away from the hollow conical member 42.

The pressure switch 25 includes a mechanical micro-switch 52. [symbol] The micro-switch 52 includes a rectangular body 55, an offset lever 57, and a single throw and single pole (STSP) switch 59, three electrical terminals 62. The STSP switch 59 includes an on/off button 65. One end of the offset lever 57 is movably attached to the rectangular body 55. A middle portion 57a of the offset lever 57 is placed next to the on/off button 65. Two ends of the STSP switch 59 are electrically connected to two of the electrical terminals 62. The offset lever 57 is placed adjacent to one end of the actuator member 44. The electrical terminals 62 are electrically connected to the electronic testing unit 28.

The test probe 16 includes a metal tube 68 with a reflective inner surface 70 together with a protective shell 74. A first end 68a of the metal tube 68 protrudes from the head portion 38 and is placed outside the head portion 38. The protective shell 74 surrounds a second 68b of the metal tube 68 and it touches the second end 68b of the metal tube 68. The protective shell 74 also provides a cavity 76 that is placed next to the second end 68b of the metal tube 68.

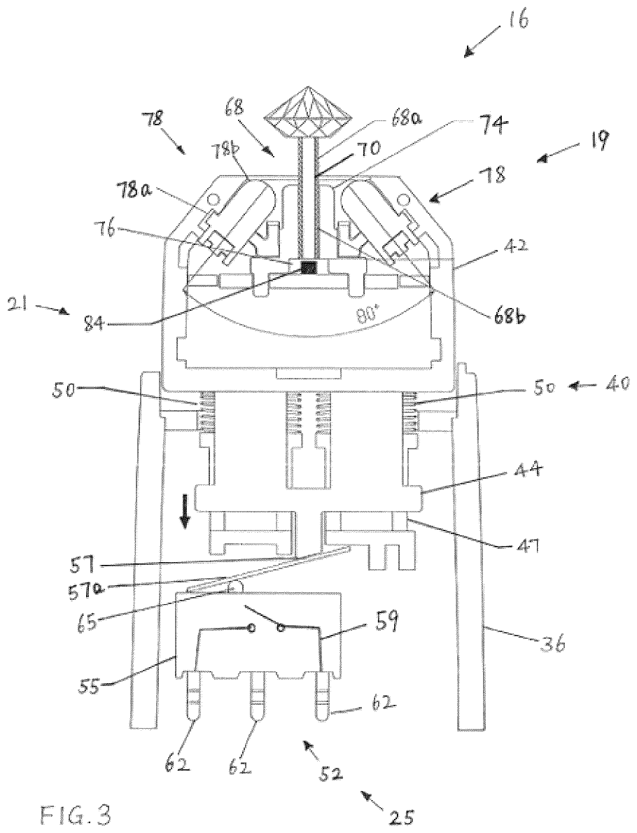

As seen in FIG. 3, the light module 19 includes two light sources 78. The light sources 78 are positioned near to the test probe 16 and they are also placed around the test probe 16 in a symmetrical manner. The light sources 78 are placed opposite to each other. Each light source 78 includes a cylindrical body 78a and a semi-spherical part 78b that is placed at one end of the cylindrical body 78a. The cylindrical body 78a is inclined at an angle of about 40 degrees with respect to the longitudinal axis of the metal tube 68 and it is pointing towards a predetermined location that is positioned near to the first end 68a of the metal tube 68. The light sources 78 are electrically connected to the electronic testing unit 102 via current limiting resistors 96. Each light source 78 includes one ultraviolet (UV) Light Emitting Diode (LED).

The photodetector 21 includes a photodiode 84. The photodiode 84 is placed adjacent to the second end 68b of the metal tube 68 and it is placed inside the cavity 76 that is formed by the protective shell 74. The photodiode 84 is also positioned along a longitudinal axis of the metal tube 68. The size of the photodiode 84 is comparable with the size of a diameter of the second end 68b of the metal tube 68.

The photodetector 21 has a peak detection sensitivity that corresponds with the wavelength of the ultraviolet light rays from the light sources 78. The photodetector 21 is also electrically connected to the electronic testing unit 28.

The chamber, which is formed by the protective shell 74, acts to allow only the reflected light rays from the metal tube 68 to reach the photodetector 21 while preventing other light rays from reaching the photodetector 21.

The display unit 30 includes multiple indicator lights 89 together with a low battery indicator 108. The indicator lights 89 and the low battery indicator 108 are disposed on an outer surface of the hollow body portion 36 of the handheld casing 13. The display unit 30 is electrically connected to the electronic testing unit 28.

The buzzer 92 is placed inside the hollow body portion 36 of the handheld casing 13. The buzzer 92 is also electrically connected to the electronic testing unit 28.

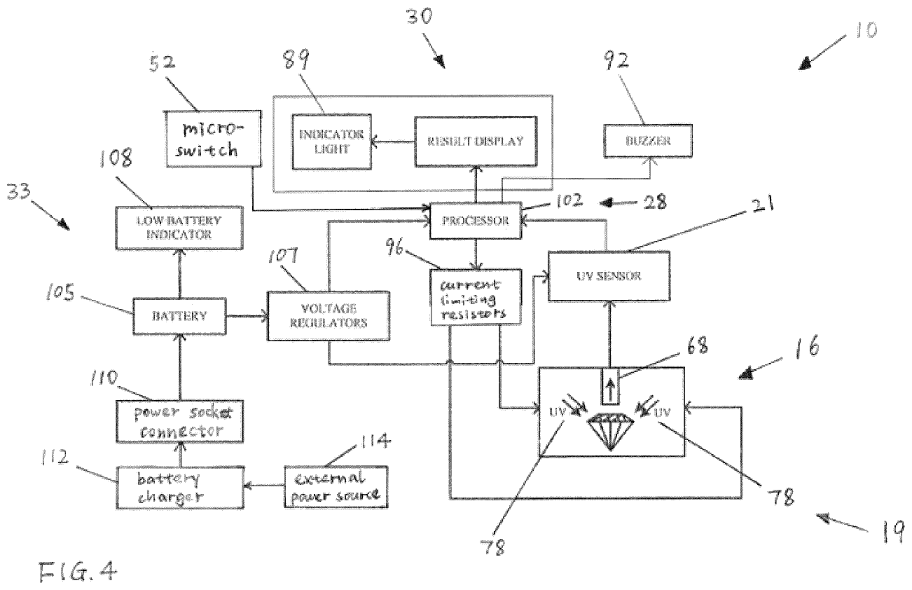

As shown in FIG. 4, the electronic testing unit 28 includes a processor unit 102. The photodetector 21 and the light sources 78 together with the indicator lights 89, the low battery indicator 108, and the buzzer 92 of the display unit 30 are electrically connected to the processor unit 102.

The power source unit 33 includes a battery module 105 with a voltage regulator 107, a power socket connector 110, and a battery charger 112. The battery module 105, and the voltage regulator 107 are placed inside the hollow body portion 36. The power socket connector 110 is partially enclosed in the hollow body portion 36 and is placed at the second end 36b of the hollow body portion 36. The battery charger 112 is adapted for electrically connecting to an external power source 114 and for electrically connecting to the power socket connector 110. The power socket connector 110 is electrically connected to the battery module 105. The battery module 105 and the voltage regulator 107 are adapted for providing electrical power to electronic components of the electronic testing unit 28. The battery module 105 includes a lithium battery that is electrically connected to contact terminals that are soldered onto a printed circuit board (PCB).

In one implementation, the metal tube 68 has a length of about 9.30 millimeter (mm).

In a special implementation, the light source 78 produces a UV light with a wavelength of about 365 nm. The photodetector 21 has a peak detection sensitivity at about 365 nm.

The indicator lights 89 may be provided by LEDs with suitably chosen colors.

Functionally, the gemstone testing apparatus 10 provides a way to differentiate between diamond and moissanite.

In use, a thermal conductivity test can be used to separate diamond and moissanite from all other gemstones. Thereafter, the gemstone testing apparatus 10 can be used to differentiate between diamond and moissanite.

The gemstone testing apparatus 10 is intended to be held by a user such that the first end 68a of the metal tube 68 is placed on the surface of a specimen.

The user then presses the metal tube 68 against the specimen. The hollow conical member 42 with the actuator member 44 then moves into the body portion 36, along the longitudinal axis of the elongated body portion 36 by a substantially small distance. The hollow conical member 42 with the actuator member 44 also move towards the micro-switch 52. This movement acts to compress the springs 50.

The actuator member 44 afterward pushes the offset lever 57 of the micro-switch 52 such that the offset lever 57 pushes the on/off button 65 of the micro-switch 52 into the rectangular body 55 of the micro-switch 52.

The micro-switch 52 can be placed in a closed and a normally open position. The above pushing of the on/off button 65 acts to place the micro-switch 52, from the open position, to the closed position. The micro-switch 52 also acts to provide a switch position signal to the processor unit 102.

The current limiting resistor 96 acts to regulate electrical current to the light sources 78, when the light sources 78 are activated by the processor unit 102.

The activated light sources 78 produce ultraviolet light rays. The ultraviolet light rays are intended for illuminating a specimen, which is placed near to the test probe 16, when it is activated by the processor unit 102. The light ray is also called light for short.

The ultraviolet light rays have a predetermined fixed wave-length that is within a predetermined UV light spectrum band.

The ultraviolet light rays can also have different wavelengths that are within the predetermined UV light spectrum band. In one implementation, the predetermined UV light spectrum band is between about 315 nm and about 400 nm.

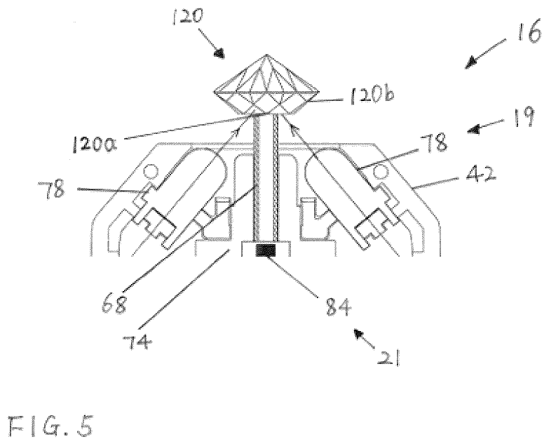

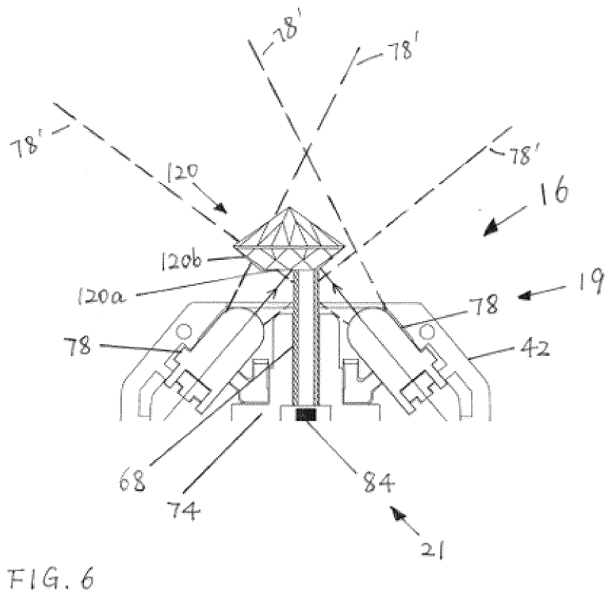

As seen in FIGS. 5 and 6, the two light sources 78 allow a table 120a of the specimen 120 to receive light from either one or two of the light sources 78, when the metal tube 68 is placed at different parts of the table 120a, such as an edge of the table 120a.

In practice, the size of the metal tube 68 is often smaller than the size of the table 120a of the specimen 120. Because of this, a user may place the metal tube 68 at different parts of the table 120a of the specimen 120. The metal tube 68 can be placed near to a center location or be placed at an edge of the table.

The table 120a of the specimen 120 refers to a flat facet on the top of a gemstone specimen 120. One example of the specimen 120 is a diamond or moissanite. The flat facet is usually the largest facet of a cut specimen.

When the metal tube 68 is placed substantially near or at the center location of the table 120a of the specimen 120, the table 120a of the specimen 120 would receive light emitted from both light sources 78, as illustrated in FIG. 5.

When the metal tube 68 is not placed substantially near the center location of the table 120a of the specimen 120, such as the edge of the specimen 120, the table 120a of the specimen 120 would still receive light emitted from one of the two light sources 78. This is illustrated by ray lights with borders 78′ in FIG. 6.

In short, the two light sources allow the table 120a of the specimen 120 to receive light rays from the light sources even when the metal tube 68 is placed at different parts of the table 120a of the specimen 120.

This is different from other gemstone testers with a test probe tube and with just one single light source.

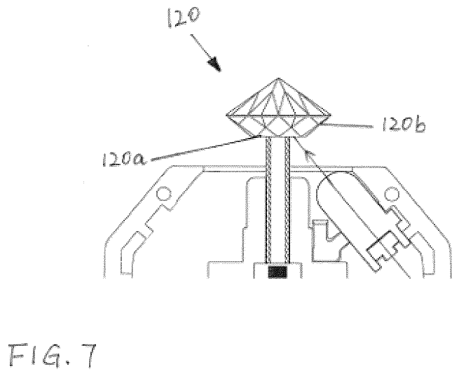

When the test probe tube is placed near or at a center location of a table 120a of a specimen 120, the table 120a would receive light rays from the single light source, as shown in FIG. 7.

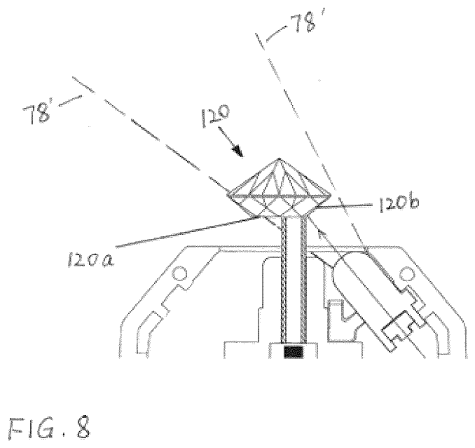

When the probe tube is placed at an edge of the table 120a of the specimen 120, only a side facet 120b of the specimen 120 may receive light rays from the single light source, as shown in FIG. 8. In other words, no light rays or little light rays are directed onto the table 120a of the specimen.

The specimen 120 may then not receive enough light rays for testing the specimen 120. This then degrade or affect the testing to the specimen 120.

Referring to the specimen 120, it can include a moissanite or a diamond. If the specimen 120 includes a moissanite, the moissanite would absorb these light rays from the light sources 78. In other words, no light rays are reflected from the moissanite. If the specimen 120 includes a diamond, the diamond would reflect the light rays or reflect part of the light rays from the light sources 78.

The metal tube 68 acts as a light guide to receive the light rays reflected from the specimen 120.

In detail, the second end 68b of the metal tube 68 receives the light rays reflected from the specimen 120. The inner surface of the metal tube 68 then reflects these light rays without absorbing these light rays. The inner surface also directs these light rays to the second end 68b of the metal tube 68 and towards the photodetector 21.

Referring to the protective shell 74, it provides a structural support for the two light sources 78 and for the metal tube 68, preventing them from moving.

The photodetector 21 detects and measures intensity of light rays being reflected from the specimen. The photodetector 21 then sends the light measurements to the processor unit 102.

The indicator lights 89 receives an electrical signal regarding a gemstone test result from the processor unit 102 and then emits a corresponding light for showing the gemstone test result to a user. As an example, the indicator lights 89 activates a first LED for showing that the gemstone testing apparatus 10 detects a diamond. The indicator lights 89 activates a second LED for showing that the gemstone testing apparatus 10 detects a moissanite.

The buzzer 92 also receives a signal from the processor unit 102 and generates a corresponding audio sound in accordance to the signal. The buzzer 92 produces a continuous beeping sound when the gemstone testing apparatus 10 detects a diamond. The buzzer 92 produces a short intermittent beeping sound when the gemstone testing apparatus 10 detects a moissanite.

After the indicator lights 89 emits a light for showing the gemstone test result to the user, the user can remove the metal tube 68 away from the specimen 120.

The coil torsion springs 50 then pushes the hollow conical member 42 and the actuator member 44 away from the micro-switch 52.

The actuator member 44 afterward does not push and does not contact the offset lever 57 of the micro-switch 52.

The micro-switch 52 then returns to its open position from its [symbol] closed position. The micro-switch 52 then provides a switch position signal to the processor unit 102.

The battery module 105 supplies electrical power to the light sources 78, to the electronic testing unit 28, and to the display unit 30.

The voltage regulator 107 allows the battery module 105 to provide an output voltage with a constant voltage level.

The battery charger 112 together with the power socket connector 110 is used for connecting to an external power source 114. The connecting allows the external power source 114 to charge the battery module 105. The charging provides electrical energy to the battery module 105.

The processor unit 102 includes a program or instructions to receive a switch position signal from the micro-switch 52. After this, the processor unit 102 activates the light sources 78 according to the received switch position signal. The processor unit 102 later also receives light intensity measurements from the photodetector 21 after a predetermined period. The processor unit 102 then determines a gemstone test result in accordance to the light intensity measurements.

The processor unit 102 transmits an electrical signal regarding the determined gemstone test result to the indicator lights 89. The processor unit 102 can also send a corresponding signal to the buzzer 92.

The processor unit 102 monitors the output voltage of the battery module 105 and provides an alert signal to the low battery indicator 108. The low battery indicator 108 then emits a corresponding light to the user.

The handheld casing 13 acts to contain and protect parts of the gemstone testing apparatus, including the light module 19, the test probe 16, the power source unit 33 and the electronic testing unit 28 and the display unit 30.

The hollow conical member 42 of the head portion 38 is used for containing and protecting the light sources 78, the test probe 16, the photodetector 21 and a part of the electronic testing unit 28.

The elongated hollow body portion 36 is provided for containing and protecting the pressure switch, the display unit 30,the power source unit 33 and a part of the electronic testing unit 28.

The gemstone testing apparatus 10 provides several benefits.

The two light sources 78 enable the table 120a of the specimen 120 to receive sufficient light rays from the light sources 78, even when the metal tube 68 is placed at different parts of the table 120a, such as an edge of the table 120a.

In use, the specimen 120 is often small. Because of this, a user may place the metal tube 68 at different parts of the table 120a of the specimen 120. For example, the metal tube 68 can be placed near to a center location of the table 120a of the specimen 120. It can also be placed at an edge of the table 120a. In spite of this, the two light sources 78 enable the specimen 120 to receive enough light rays for testing the specimen.

The length of the metal tube 68 prevents the metal tube 68 from being easily bent. A distance between the first end 68a of the metal tube 68 and the light sources 78 is also short enough to enable light rays from the light sources 78 to reach the specimen 120 with no or little loss of light rays, thereby not reducing light intensity.

In a general sense, the indicator lights 89 can be replaced by a display panel, such as a color or a monochrome screen dis-play, which can be provided by a Liquid Crystal Display (LCD) or an Organic Light-Emitting Diode (OLED) display.

The handheld casing 13 can include a catch which allows an external cap 121 to be attached to the handheld casing 13 using a snap-fit mechanism. The external cap 121 is used for protecting the test probe 16 from being damaged.

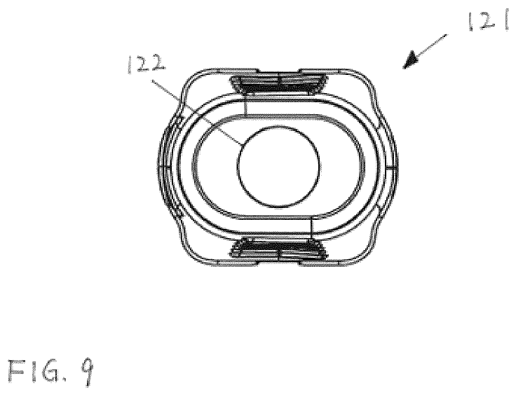

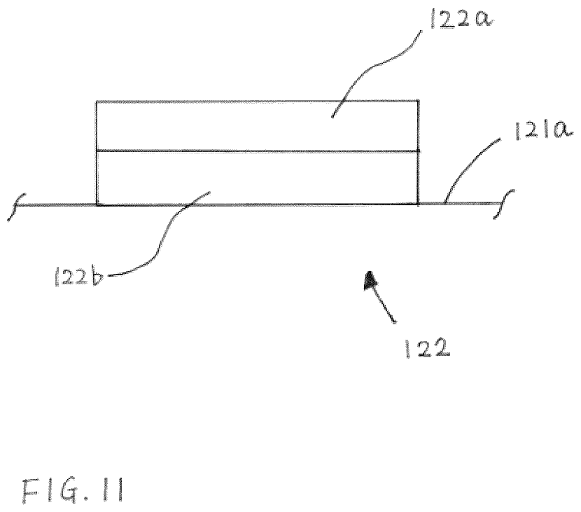

As shown in FIGS. 9 and 11, the external cap 121 can include a gemstone test reference tablet 122. The gemstone test reference tablet 122 is provided on an outer surface 121a of the external cap 121 for easy access. The gemstone test reference tablet 122 includes a layer 122a of transparent material and a layer 122b of reflective material. The transparent material layer 122a is placed next to the reflective material layer 122b while the reflective material layer 122b is placed next to the outer surface 121a of the external cap 121.

A user may use the gemstone test reference tablet 122 to check functions of the gemstone testing apparatus 10. The user presses the first end 68a of the metal tube 68 of the test probe 16 of the gemstone testing apparatus 10 against the gemstone test reference tablet 122. The reflective material layer 122b then acts to reflect light from the light sources 78 of the gemstone testing apparatus 10, just like a diamond, while the transparent material layer 122a acts to protect the reflective material layer 122b.

The metal tube 68 can be replaced by a light guide, such as a hollow tube, wherein an inner surface of the hollow tube is coated with a reflective layer.

The gemstone testing apparatus 10 can include three or more light sources, instead of just two light sources. These light sources are placed around the metal tube 68 in a symmetric manner. Each of the light sources can be positioned at a pre-determined angle with respect to the longitudinal axis of the gemstone testing apparatus 10. The multiple light sources can allow production of light rays with a higher intensity for illuminating the specimen 120.

The light sources can be replaced by a ring light enclosing the test probe 16. The ring light can be configured to emit light rays that are directed to a location near the first end 68a of the metal tube 68. The ring light can also enable production of light rays with a higher intensity for illuminating the specimen 120.

The processor unit 102 includes a peripheral module that includes a timer. The timer can be programmed or instructed to switch off the electrical power of the gemstone testing apparatus 10, when the electronic testing unit 28 is inactive for a predetermined period. Put differently, the gemstone testing apparatus 10 is automatically powered off when it is not in use for a predetermined period to conserve or save power.

The display unit 30 can include an electrical power indicator for showing that the electronic testing unit 28 is powered on.

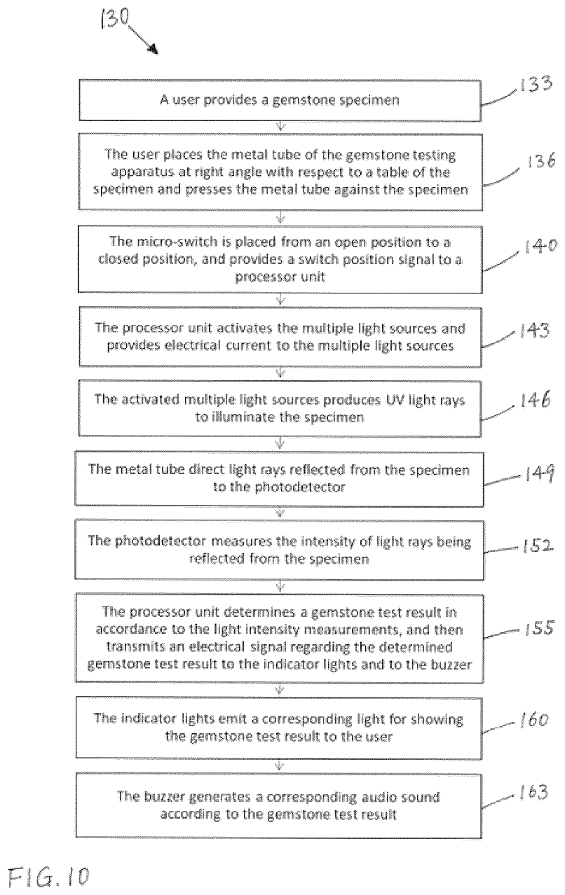

FIG. 10 shows a flow chart 130 of a method of operating the gemstone testing apparatus 10.

The flow chart 130 includes a step 133 of a user providing a specimen 120.

The user then presses the metal tube 68 of the gemstone testing apparatus 10 against the specimen, in a step 136. The metal tube 68 is placed such that it is about at right angle with respect to the table 120a of the specimen 120.

This later causes the micro-switch 52 to be placed, from its open position, to the closed position, in a step 140. The micro-switch 52 also provides a switch position signal to the processor unit 102.

The processor unit 102 activates the multiple light sources and provides electrical current to the multiple light sources 78, in a step 143.

The activated multiple light sources 78 afterward produces ultraviolet light rays to illuminate the specimen 120, in a step 146.

The metal tube 68 subsequently directs these light rays to the second end 68b of the metal tube 68 and to the photodetector 21, in a step 149.

The photodetector 21 then measures the intensity of light rays being reflected from the specimen 120, in a step 152.

The processor unit 102 then determines a gemstone test result in accordance to the light intensity measurements. The processor unit 102 transmits an electrical signal regarding the determined gemstone test result to the indicator lights 89 and to the buzzer 92, in a step 155.

The indicator lights 89 receives the electrical signal regarding a gemstone test result from the processor unit 102 and then emits a corresponding light for showing the gemstone test result to the user, in a step 160.

The buzzer 92 also receives the electrical signal from the processor unit 102 and then produces a corresponding audio sound according to the gemstone test result, in a step 163.

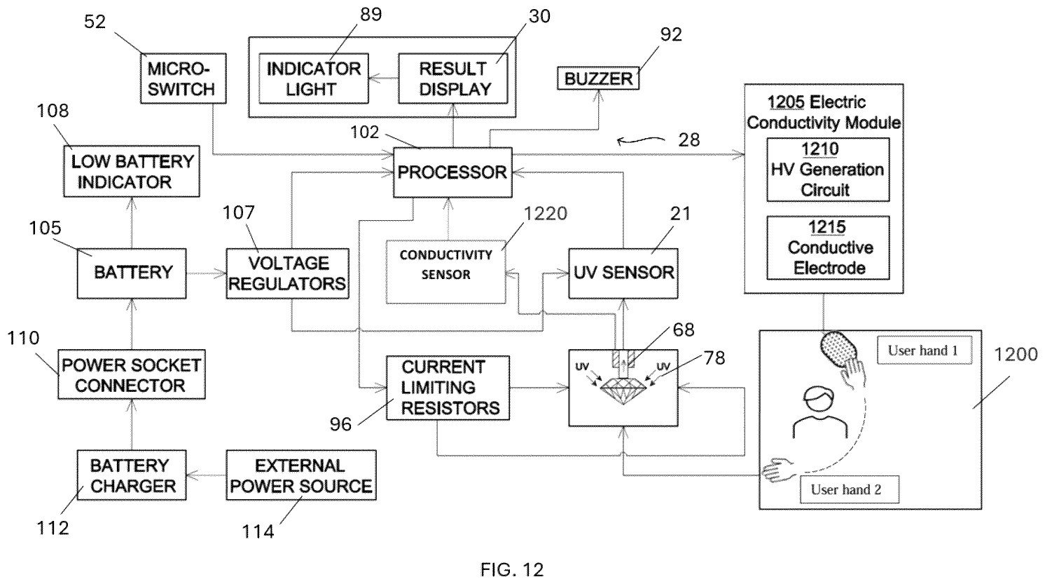

FIG. 12 illustrates an electronic block diagram of a gemstone testing apparatus 1200, depicting the components necessary for conducting both electrical conductivity testing and ultraviolet light-based gemstone evaluation.

The gemstone testing apparatus 1200 includes an elongated handheld body, incorporating a test probe 16 at the body proximal end. The apparatus further includes a light module including ultraviolet light sources 78, which emit ultraviolet light towards a gemstone specimen. The light reflected from the gemstone is directed through a tubular optical transmission channel, integrated into the test probe, and received by a photodetector 21. The photodetector is optically coupled to the tubular channel and configured to sense the reflected ultraviolet light for analysis.

A processor 102 is included in the apparatus to control various functions. It is operably connected to the light sources 78, the photodetector 21, and a display unit 30, which displays the gemstone test result. The processor is also coupled to an electronic conductivity module 1205 to conduct electrical tests. The electronic conductivity module includes a high-voltage generation circuit 1210 and a conductive electrode 1215. The conductive electrode transmits high-voltage direct current to the gemstone specimen to measure its conductivity. The conductivity sensor 1220 detects the resulting electrical signals transmitted through the gemstone specimen.

The gemstone testing apparatus 1200 also includes a power source unit. This consists of a battery module 105, a voltage regulator 107, and a power socket connector 110, which enables charging through an external power source 114 or a battery charger 112. A low battery indicator 108 alerts the user when the battery level is insufficient. Current-limiting resistors 96 regulate electrical flow to the components.

A micro-switch 52 serves as an actuator to activate the electronic testing system, which includes the ultraviolet light sources and the conductivity module. A buzzer 92 provides an audible alert upon detecting certain test results, such as identifying a gemstone as man-made.

For electrical conductivity testing, the conductive electrode 1215 may be in contact with a loose gemstone or a mounted stone. The gemstone specimen can be irradiated with ultraviolet light from the light sources 78 prior to conductivity testing to reduce electrical resistance, which is processed by the conductivity sensor 1220 and analyzed by the processor 102 to determine the type of gemstone.

In this embodiment, the gemstone testing apparatus 1200 integrates functionalities such as UV-based analysis and conductivity measurements to distinguish between gemstones like moissanite and diamonds. For instance, moissanite conduct electricity while colorless diamonds act as insulators. The dissipation rate of the direct current is used to differentiate between gemstone types.

An indicator light 89 is also included to provide visual feedback during testing.

In FIG. 12, User hand 2 operates the gemstone testing apparatus 1200 by interacting with the gemstone specimen 120 or the conductive electrode 1215. During the electrical conductivity test, User hand 2 comes into contact with either the gemstone specimen 120 or a conductive component of the gemstone mounting, such as a metal part of a ring. This contact establishes a pathway for high-voltage direct current generated by the high-voltage generation circuit 1210 to pass through the gemstone specimen 120, enabling the conductivity sensor 1220 to detect the transmitted signal.

User hand 1 holds and stabilizes the elongated handheld body of the gemstone testing apparatus 1200 during operation. The user applies grip pressure on the device, including on areas integrated with conductive components such as the conductive electrode 1215. This ensures proper electrical conduction and mechanical stability while the apparatus conducts both ultraviolet light- based testing and electrical conductivity testing.

By using both hands in conjunction, User hand 2 interacts with the gemstone specimen or its mounting to facilitate electrical conductivity measurements, while User hand 1 securely.

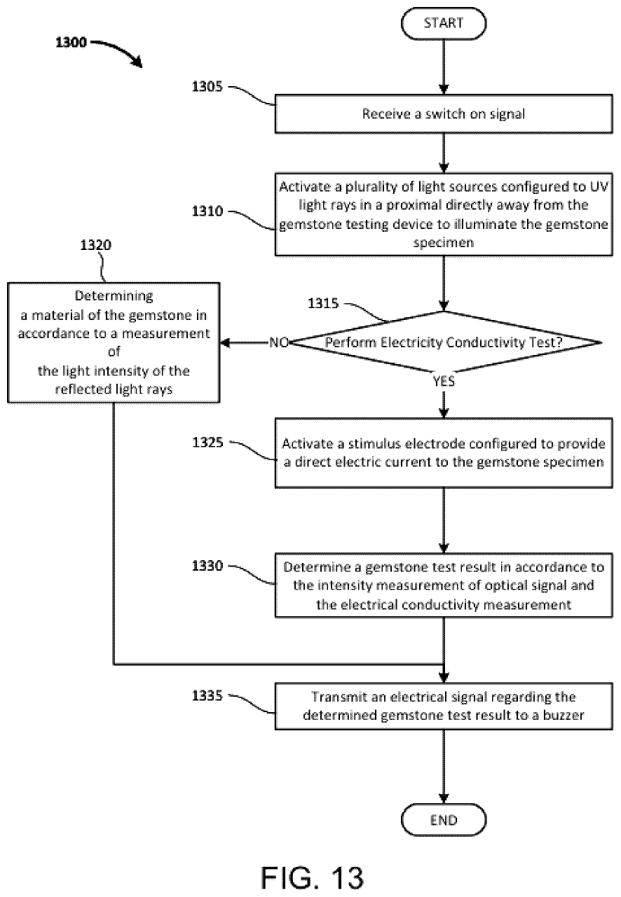

FIG. 13 illustrates a flowchart 1300 depicting the operation of the gemstone testing apparatus 1200 for evaluating a gemstone specimen 120 using both ultraviolet light intensity measurements and electrical conductivity testing.

The process begins when the apparatus 1200 receives a switch-on signal in step 1305. This signal activates the electronic components of the apparatus, including the processor 102 and associated circuits. The apparatus then activates a plurality of ultraviolet light sources 78, configured to emit ultraviolet light rays in a proximal direction away from the gemstone testing apparatus 1200 towards the gemstone specimen 120, as shown in step 1310. The ultraviolet light illuminates the gemstone specimen, and the reflected light is detected by a photodetector 21 via a tubular optical transmission channel in the test probe 16.

The apparatus determines whether to proceed with an electrical conductivity test in step 1315. The gemstone testing apparatus 1200 decides whether to proceed with an electrical conductivity test based on its programmed operation logic managed by the processor 102. In FIG. 13, this decision is represented in step 1315, where the apparatus evaluates the need for an electrical conductivity test after receiving data from the photodetector 21.

Initially, the apparatus activates the ultraviolet light sources 78 to illuminate the gemstone specimen 120. The photodetector 21 measures the intensity of the reflected ultraviolet light rays. If the optical measurement alone is sufficient to determine the material of the gemstone, the apparatus concludes the evaluation without performing the electrical conductivity test, as shown in step 1320. This determination uses data from the photodetector 21 to evaluate the light properties of the gemstone specimen.

However, if the optical data does not provide conclusive results about the gemstone's material, the apparatus proceeds with the electrical conductivity test. The processor 102 activates the conductive electrode 1215 in step 1325. The stimulus electrode applies a direct electric current generated by the high-voltage generation circuit 1210 to the gemstone specimen 120. The current flows through the specimen and is detected by the conductivity sensor 1220. The apparatus uses both the optical intensity measurements and the electrical conductivity measurements to determine the gemstone test result in step 1330.

The processor 102 then generates an electrical signal representing the test result and transmits it to a buzzer 92, as described in step 1335. The buzzer produces an audible signal, providing feedback to the user about the gemstone's characteristics. This process allows the apparatus to distinguish between gemstones such as moissanite, which conducts electricity, and colorless diamonds, which act as insulators.

This flowchart demonstrates the combined use of ultraviolet light analysis and electrical conductivity testing to ensure accurate gemstone identification. The steps are controlled and executed by the electronic testing unit 28, which integrates optical and electrical components for seamless operation.

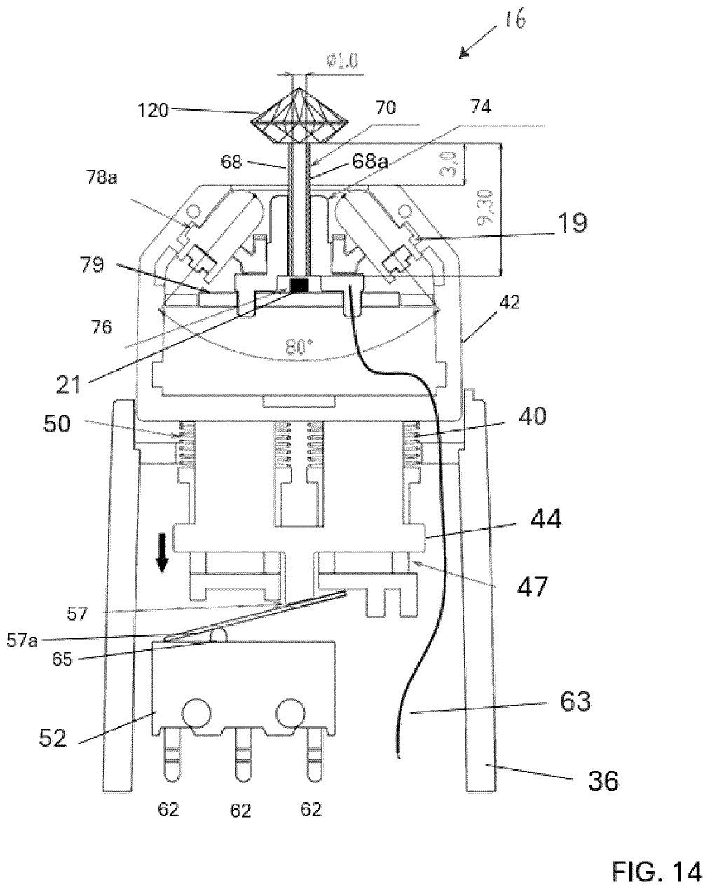

FIG. 14 illustrates the internal configuration of the gemstone testing apparatus 10, emphasizing the light module 19, optical testing components, and their integration with the electrical testing system. The figure shows the detailed arrangement of components for performing ultraviolet light and electrical conductivity-based gemstone testing.

The light module 19 includes two ultraviolet light sources 78 positioned symmetrically around the test probe 16 and opposite each other. Each light source 78 consists of a cylindrical body 78a and a semi-spherical part 78b located at one end of the cylindrical body. The cylindrical bodies 78a are inclined at an angle of approximately 40 degrees with respect to the longitudinal axis of the metal tube 68. This arrangement ensures that the ultraviolet light emitted by the light sources is directed towards a predetermined location near the first end 68a of the metal tube 68. Each light source 78 includes a UV Light Emitting Diode (LED) and is electrically connected to the electronic testing unit 102 via current limiting resistors 96.

The reflected ultraviolet light from the gemstone specimen 120 passes through the metal tube 68 and reaches the photodetector 21. The photodetector 21 includes a photodiode 84 positioned inside the cavity 76 formed by the protective shell 74. The photodiode 84 is aligned along the longitudinal axis of the metal tube 68 and is adjacent to the second end 68b of the metal tube. The photodiode 84 has a size comparable to the diameter of the second end 68b of the metal tube and is designed with a peak detection sensitivity that corresponds to the wavelength of the ultraviolet light emitted by the light sources 78. The photodetector 21 is electrically connected to the electronic testing unit 28.

The protective shell 74 forms a chamber around the photodiode 84. This chamber allows only the reflected ultraviolet light from the metal tube 68 to reach the photodetector 21 while blocking other stray light rays, ensuring accurate light intensity measurements.

The mechanical assembly includes a spring support unit 40 with coil torsion springs 50 that stabilize the positioning of the test probe 16. The actuator member 44 interacts with a support member 47 to control the movement of the test probe. The mechanical micro-switch 52 is connected to the actuator and can be activated by the offset lever 57, which activates the on/off button 65. Electrical terminals 62 connect the micro-switch 52 to the electronic circuitry.

The light module 19, in conjunction with the optical and electrical components, ensures precise detection of both ultraviolet light intensity and electrical conductivity. These integrated systems enable the apparatus to evaluate the gemstone specimen 120 and determine its material properties.

The figure introduces the printed circuit board (PCB) 79, which electrically connects the ultraviolet light sources 78 to the electronic testing unit 28. The PCB 79 ensures precise control and reliable operation of the UV LED housed within the cylindrical bodies 78a of the light sources 78. This component adds an essential layer of integration, supporting the overall optical testing process.

The figure also shows the wire 63, which connects the metal tube 68 to the conductivity sensor 1220. This wire transmits the electrical signals generated when the high-voltage generation circuit 1210 applies a direct electric current to the gemstone specimen 120 via the conductive electrode 1215. The wire 63 plays a critical role in the conductivity testing function by establishing a pathway for the signals to reach the conductivity sensor 1220. This connectivity enables the apparatus to measure the electrical properties of the gemstone specimen with accuracy.

When the high-voltage generation circuit 1210 applies a direct current through the conductive electrode 1215 to the gemstone specimen, the electrical signal generated as a result of the current passing through the specimen is transmitted to the conductivity sensor 1220 via the wire 63. This signal contains information about the gemstone's ability to conduct electricity, which is a distinguishing property between gemstones like moissanite and diamonds.

The wire 63 ensures an efficient and reliable transfer of the electrical signal from the metal tube 68 to the conductivity sensor 1220, minimizing signal loss or interference. The conductivity sensor 1220 processes the signal received through the wire 63 and sends the data to the processor 102 for further analysis. The processor then evaluates the conductivity data, often in combination with ultraviolet light intensity measurements, to determine the material properties of the gemstone specimen 120.

By integrating the wire 63, the apparatus ensures a seamless electrical pathway between the metal tube 68 and the conductivity sensor 1220, enabling accurate and precise measurements of electrical conductivity during the gemstone testing process.

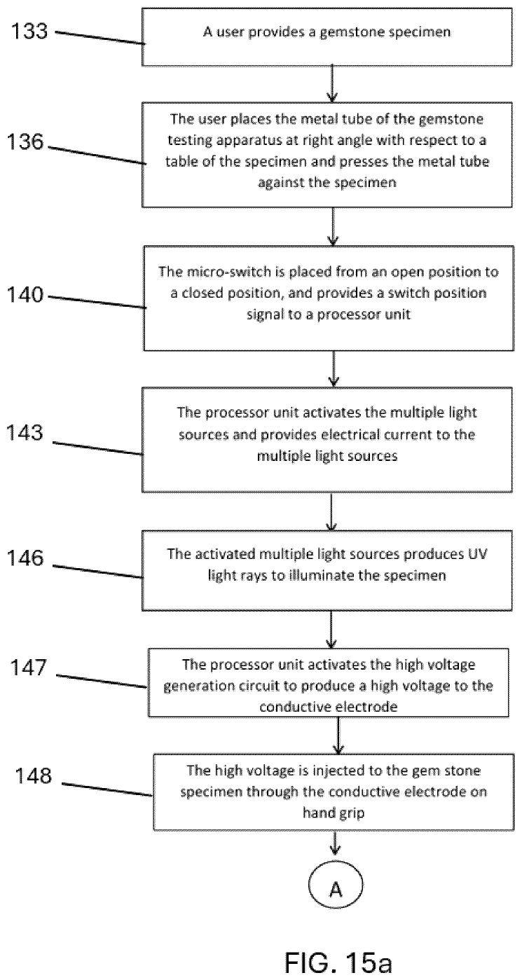

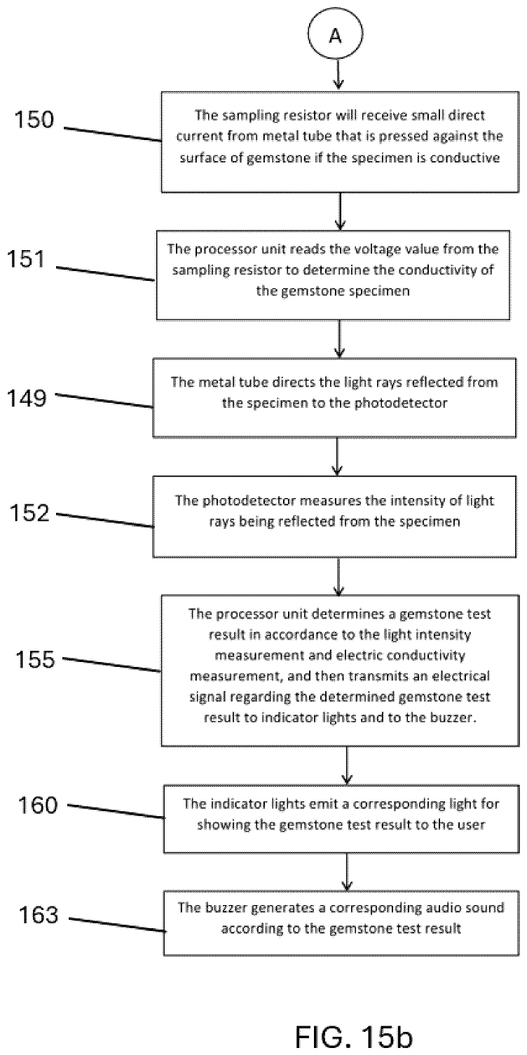

FIG. 15a and FIG. 15b illustrate a flow chart 130 depicting the method of operating the gemstone testing apparatus 10 to evaluate a gemstone specimen 120. The process begins with step 133, where the user provides a gemstone specimen 120 for testing.

In step 136, the user places the metal tube 68 of the apparatus 10 against the gemstone specimen 120, positioning it at an approximately right angle to the table of the specimen 120a. The user presses the metal tube 68 firmly against the specimen to initiate the testing process. This action triggers the micro-switch 52, as shown in step 140, which moves from its open position to a closed position. The micro-switch 52 then sends a switch position signal to the processor unit 102 to activate the electronic testing components.

Following this, in step 143, the processor unit 102 activates the multiple ultraviolet light sources 78 and supplies electrical current to them. The light sources 78 subsequently emit ultraviolet light rays to illuminate the gemstone specimen 120, as described in step 146.

At step 147, the processor unit 102 activates the high-voltage generation circuit 1210, which generates a high voltage that is delivered to the conductive electrode 1215. In step 148, the conductive electrode 1215 injects the high voltage into the gemstone specimen 120. If the specimen is conductive, a small direct current flows through the metal tube 68 and is received by the sampling resistor, as depicted in step 150. The processor unit 102 reads the voltage value from the sampling resistor in step 151, enabling it to determine the conductivity of the gemstone specimen 120.

In parallel with the electrical conductivity measurements, the metal tube 68 directs the reflected ultraviolet light rays from the gemstone specimen to the photodetector 21, as described in step 149.

In step 152, the photodetector 21, which includes a photodiode 84, measures the intensity of the ultraviolet light rays reflected from the gemstone specimen 120. These measurements are sent to the processor unit 102. The processor unit 102 then processes the light intensity measurements to determine the gemstone test result, as described in step 155. Additionally, if the gemstone specimen is conductive, as indicated by the flow of direct current detected at the metal tube 68, the processor unit 102 evaluates the electrical conductivity of the specimen using signals received from the conductivity sensor 1220. Based on the analysis, the processor unit 102 transmits an electrical signal containing the gemstone test result to the indicator lights 89 and the buzzer 92.

In step 160, the indicator lights 89 display a corresponding light to visually convey the test result to the user. Simultaneously, in step 163, the buzzer 92 produces an audible signal to indicate the test result, providing the user with additional feedback.

The flow chart in FIG. 15a and FIG. 15b demonstrates the combined use of ultraviolet light intensity measurements and electrical conductivity testing to accurately evaluate and identify the gemstone specimen 120. This process ensures a reliable and efficient operation of the gemstone testing apparatus 10.

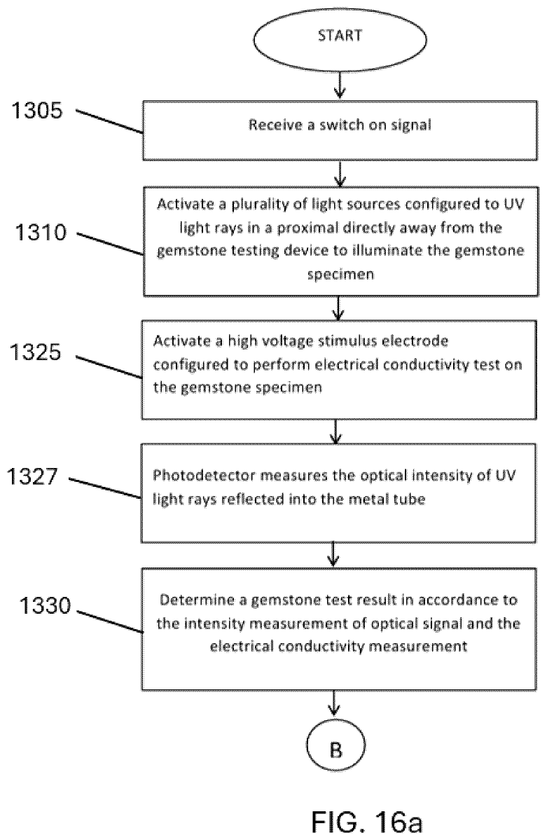

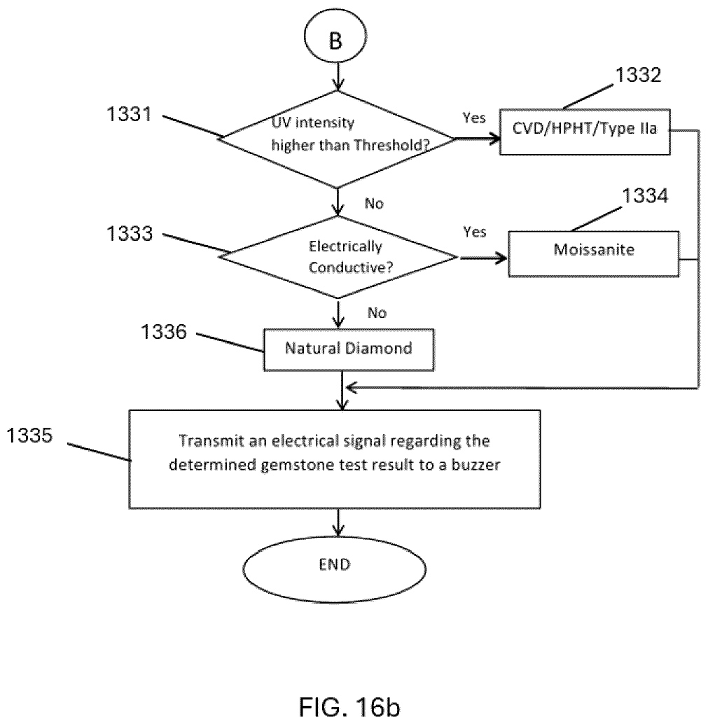

FIG. 16a and FIG. 16b illustrate a flowchart representing an exemplary embodiment of a method for operating the gemstone testing apparatus 10. This method may advantageously improve the precision of gemstone classification. Steps 1327, 1331, 1332, 1333, and 1334 are included for differentiating gemstones based on ultraviolet light intensity and electrical conductivity. The flowchart is divided into two parts, FIG. 16a and FIG. 16b, with a “B” connector indicating the continuity of the process between the figures.

In FIG. 16a, the process begins with step 1305, where the apparatus receives a switch-on signal, initiating the operation. In step 1310, a plurality of ultraviolet light sources 78 are activated to emit light rays in a proximal direction towards the gemstone specimen 120, illuminating it. Step 1325 involves activating the high-voltage stimulus electrode 1215, which performs an electrical conductivity test by delivering direct current to the gemstone specimen 120. The step 1327, measures the optical intensity of the ultraviolet light rays reflected into the metal tube 68 using the photodetector 21. The addition of this step improves the accuracy of optical measurements and allows for precise analysis of the reflected light.

In step 1330, the processor unit 102 determines the gemstone test result based on the combination of ultraviolet light intensity measurements and electrical conductivity data. The flowchart then proceeds to FIG. 16b, as indicated by the “B” connector.

In FIG. 16b, the process incorporates further refinements through additional steps. Step 1331, which replaces step 1320 from FIG. 13, evaluates whether the ultraviolet light intensity measured in step 1327 is higher than a predetermined threshold. If the intensity is above the threshold, the gemstone specimen is classified as a CVD diamond, HPHT diamond, or Type IIa diamond in step 1332. If the intensity is below the threshold, the method proceeds to step 1333, which replaces step 1315 from FIG. 13, to determine if the gemstone specimen is electrically conductive.

If the specimen is electrically conductive, step 1334 classifies it as moissanite. If the specimen is not electrically conductive, it is classified as a natural diamond, as shown in step 1336. These exemplary steps 1332, 1334, and 1336 may, in some examples, enhance the classification process by clearly defining the outcomes based on ultraviolet intensity and conductivity results.

Finally, in step 1335, the processor unit 102 transmits an electrical signal corresponding to the gemstone test result to the buzzer 92, which generates an audible signal to inform the user of the classification result. This concludes the process.

In an illustrative aspect, a hand-held gemstone testing apparatus may include an elongated handheld body extending along a longitudinal axis from a body proximal end to a body distal end. For example, the hand-held gemstone testing apparatus may include a test probe disposed at the body proximal end and including a tubular optical transmission channel extending along the longitudinal axis from a tubular channel proximal end to a tubular channel distal end, wherein the tubular channel proximal end may be configured to engage with a gemstone test specimen for gemstone testing.

For example, the hand-held gemstone testing apparatus may include a light module including a UV light source at the body proximal end wherein the light module may be configured to emit UV light in a proximal direction away from the handle body towards the gemstone test specimen when the gemstone test specimen may be engaged with the tubular channel proximal end. For example, the hand-held gemstone testing apparatus may include a photodetector in optical communication with the tubular optical transmission channel and configured to receive the UV light emitted from the light source after the UV light has reflected from the gemstone test specimen as the gemstone test specimen may be engaged with the tubular channel proximal end.

For example, the hand-held gemstone testing apparatus may include an electronic testing system included in the handheld body, operably coupled to the photodetector, an electronic circuitry, and the light module, configured to activate the light source and the electronic circuitry. For example, the hand-held gemstone testing apparatus may include a display interface disposed at an external surface of the handheld body and configured to display a gemstone test result generated by the electronic testing system when in operation. For example, the hand-held gemstone testing apparatus may include a cover to protect the test probe.

For example, the electronic circuitry may include a current transmitting electrode configured to conduct an electric current through the gemstone test specimen as the gemstone test specimen may be engaged with the tubular channel proximal end. For example, the electronic circuitry may include an electric probe configured to sense signals transmitting through the gemstone test specimen. For example, the electronic testing system may be operably coupled to the electric probe and the current transmitting electrode.

For example, the electric current includes a direct current. For example, the direct current may be within a range of 0-500V.

For example, the current transmitting electrode may be embodied within a handgrip disposed at the body proximal end.

For example, the electric probe may be integrated within the test probe.

For example, the test probe may be configured as both the current transmitting electrode and the tubular optical transmission channel.

For example, the signals sensed by the electric probe includes a conductivity measurement, and the electronic testing system may be configured to generate the gemstone test result as a function of an UV absorbance of the gemstone test specimen and the conductivity measurement.

In an illustrative aspect, a hand-held gemstone testing apparatus may include a handheld body extending along a longitudinal axis from a body proximal end to a body distal end. For example, the hand-held gemstone testing apparatus may include a test probe disposed at the body proximal end and including a transmission channel extending along the longitudinal axis from a channel proximal end to a channel distal end, wherein the channel proximal end may be configured to engage with a gemstone test specimen for gemstone testing.

For example, the hand-held gemstone testing apparatus may include a light module including a UV light source at the body proximal end, wherein the light module may be configured to emit UV light in a proximal direction away from the handle body towards the gemstone test specimen when the gemstone test specimen may be engaged with the channel proximal end.

For example, the hand-held gemstone testing apparatus may include a photodetector in optical communication with the transmission channel and configured to receive the UV light emitted from the light source after the UV light has reflected from the gemstone test specimen as the gemstone test specimen may be engaged with the channel proximal end.

For example, the hand-held gemstone testing apparatus may include a current transmitting electrode configured to conduct an electric current through the gemstone test specimen as the gemstone test specimen may be engaged with the channel proximal end.

For example, the hand-held gemstone testing apparatus may include an electric probe configured to sense signals transmitting through the gemstone test specimen.

For example, the hand-held gemstone testing apparatus may include an electronic testing system included in the handheld body, operably coupled to the photodetector, the electrode, and the light module, and may be configured to selectively operate activate the light source and the current transmitting electrode. For example, the hand-held gemstone testing apparatus may include a display interface disposed at an external surface of the handheld body and configured to display a gemstone test result generated by the electronic testing system when in operation.

For example, the transmission channel includes a tubular channel.

For example, the electric current includes a direct current.

For example, the direct current may be within a range of 0-500V.

For example, the current transmitting electrode includes a contact touch pad.

For example, the current transmitting electrode may be embodied within a handgrip disposed at the body proximal end.

For example, the electric probe may be integrated within the test probe.

For example, the test probe may be configured as both the current transmitting electrode and the tubular optical transmission channel.

For example, the signals sensed by the electric probe includes a conductivity measurement, and the electronic testing system may be configured to generate the gemstone test result as a function of an UV absorbance of the gemstone test specimen and the conductivity measurement. . . .

For example, the handheld body includes an elongated body.

In an illustrative aspect, a gemstone testing apparatus operating method may include, in response to a switch signal generated by engaging a gemstone specimen at a proximal end of a gemstone testing device, activate a plurality of light sources configured to UV light rays in a proximal directly away from the gemstone testing device to illuminate a gemstone specimen. For example, the gemstone testing apparatus operating method may include activate a stimulus electrode configured to provide a direct electric current through a touch pad to the gemstone specimen. For example, the gemstone testing apparatus operating method may include receive an intensity measurement signal of the UV light rays reflected from the gemstone specimen through a test probe including a tubular optical transmission channel.

For example, the gemstone testing apparatus operating method may include receive an electric current measurement signal of the electric current conducted through the gemstone specimen from the test probe. For example, the gemstone testing apparatus operating method may include determine a gemstone test result in accordance to signals received from the test probe. For example, the gemstone testing apparatus operating method may include transmit an electrical signal regarding the determined gemstone test result to a buzzer, such that a moissanite of a colorless range may be to be detected.

For example, the gemstone testing apparatus operating method may include receive an electric test disable signal. For example, the stimulus electrode may be deactivated upon receiving the electric test disable signal, and the gemstone test result may be determined based solely on the intensity measurement signal.

Although the above description contains much specificity, these should not be construed as limiting the scope of the embodiments but merely providing illustration of the foreseeable embodiments. Especially the above stated advantages of the embodiments should not be construed as limiting the scope of the embodiments but merely to explain possible achievements if the described embodiments are put into practice. Thus, the scope of the embodiments should be determined by the claims and their equivalents, rather than by the examples given.

### REFERENCE NUMBERS

10 gemstone testing apparatus
13 elongated handheld casing
16 test probe
19 light module
21 photodetector
25 pressure switch
28 electronic testing unit
30 display unit
33 power source unit
36 elongated hollow body portion
36a first end of the elongated hollow body portion
36b second end of the elongated hollow body portion
38 head portion
40 spring support unit
42 hollow conical member of the head portion
44 actuator member of the head portion
47 support member
50 coil torsion springs
52 mechanical micro-switch
55 rectangular body
57 offset lever
59 single throw and single pole (STSP) switch
62 electrical terminals
65 on/off button
68 metal tube
68a first end of the metal tube
68b second end of the metal tube
70 reflective inner surface
74 protective shell
76 cavity formed by the protective shell
78 light sources
78′ border of light rays
84 photodiode
89 indicator lights
92 buzzer
96 current limiting resistors
102 processor unit
105 battery module
107 voltage regulator
108 low battery indicator
110 power socket connector
112 battery charger
114 external power source
120 specimen
120a table of the specimen
120b side facet of the specimen
121 external cap
121a outer surface
122 gemstone test reference tablet
122a layer
122b layer
130 flow chart
133 provision of gemstone specimen
136 placement of gemstone testing apparatus
140 open to closed position of micro-switch
143 activation of light sources
146 illumination of specimen
147 activation of HV generation circuit
148 conductive electrode delivers high voltage to specimen
149 reflected light rays directed to photodetector
150 sampling resistor gets direct current
151 processing of voltage value from sampling resistor
152 measurement of reflected light rays intensity
155 determination of gemstone test result
160 indicator lights based on gemstone test result
163 buzzer feedback based on gemstone test result
1200 gemstone testing apparatus
1220 conductivity sensor
1205 electric conductivity module
1210 HV Generation Circuit
1215 conductive electrode
1300 Flowchart
1305 Receive a switch-on signal
1310 Activate a plurality of light sources
1315 Perform electricity conductivity test
1320 Determining a material of the gemstone
1325 Activate a stimulus electrode
1327 Measurement of optical intensity
1330 Determine a gemstone test result
1331 Measurement of UV intensity
1332 CVD/HPHT/Type IIa
1333 Measurement of electrical conductivity
1334 Moissanite
1335 Transmit an electrical signal to a buzzer
1336 Natural Diamond

What is claimed is:

1. A hand-held gemstone testing apparatus including:
an elongated handheld body extending along a longitudinal axis from a body proximal end to a body distal end;
a test probe disposed at the body proximal end and comprising a tubular optical transmission channel extending along the longitudinal axis from a tubular channel proximal end to a tubular channel distal end, wherein the tubular channel proximal end is configured to engage with a gemstone test specimen for gemstone testing;
a light module comprising a UV light source at the body proximal end wherein the light module is configured to emit UV light in a proximal direction away from the handle body towards the gemstone test specimen when the gemstone test specimen is engaged with the tubular channel proximal end;
a photodetector in optical communication with the tubular optical transmission channel and configured to receive the UV light emitted from the light source after the UV light has reflected from the gemstone test specimen as the gemstone test specimen is engaged with the tubular channel proximal end;
an electronic testing system comprised in the handheld body, operably coupled to the photodetector, an electronic circuitry, and the light module, configured to activate the light source and the electronic circuitry;
a display interface disposed at an external surface of the handheld body and configured to display a gemstone test result generated by the electronic testing system when in operation; and,
a cover to protect the test probe.

2. The hand-held gemstone testing apparatus of claim 1, wherein the electronic circuitry comprises:
a current transmitting electrode configured to conduct an electric current through the gemstone test specimen as the gemstone test specimen is engaged with the tubular channel proximal end; and,
an electric probe configured to sense signals transmitting through the gemstone test specimen, wherein the electronic testing system is operably coupled to the electric probe and the current transmitting electrode.

3. The hand-held gemstone testing apparatus of claim 2, wherein the electric current comprises a direct current.

4. The hand-held gemstone testing apparatus of claim 3, wherein the direct current is within a range of 0-500V.

5. The hand-held gemstone testing apparatus of claim 2, wherein the current transmitting electrode is embodied within a handgrip disposed at the body proximal end.

6. The hand-held gemstone testing apparatus of claim 2, wherein the electric probe is integrated within the test probe.

7. The hand-held gemstone testing apparatus of claim 2, wherein the test probe is configured as both the current transmitting electrode and the tubular optical transmission channel.

8. The hand-held gemstone testing apparatus of claim 2, wherein the signals sensed by the electric probe comprises a conductivity measurement, and the electronic testing system is configured to generate the gemstone test result as a function of an UV absorbance of the gemstone test specimen and the conductivity measurement.

9. A hand-held gemstone testing apparatus comprising:
a handheld body extending along a longitudinal axis from a body proximal end to a body distal end;
a test probe disposed at the body proximal end and comprising a transmission channel extending along the longitudinal axis from a channel proximal end to a channel distal end, wherein the channel proximal end is configured to engage with a gemstone test specimen for gemstone testing;
a light module comprising a UV light source at the body proximal end, wherein the light module is configured to emit UV light in a proximal direction away from the handle body towards the gemstone test specimen when the gemstone test specimen is engaged with the channel proximal end;
a photodetector in optical communication with the transmission channel and configured to receive the UV light emitted from the light source after the UV light has reflected from the gemstone test specimen as the gemstone test specimen is engaged with the channel proximal end;
a current transmitting electrode configured to conduct an electric current through the gemstone test specimen as the gemstone test specimen is engaged with the channel proximal end;
an electric probe configured to sense signals transmitting through the gemstone test specimen;
an electronic testing system comprised in the handheld body, operably coupled to the photodetector, the electrode, and the light module, and is configured to selectively operate activate the light source and the current transmitting electrode; and,
a display interface disposed at an external surface of the handheld body and configured to display a gemstone test result generated by the electronic testing system when in operation.

10. The hand-held gemstone testing apparatus of claim 9, wherein the transmission channel comprises a tubular channel.

11. The hand-held gemstone testing apparatus of claim 9, wherein the electric current comprises a direct current.

12. The hand-held gemstone testing apparatus of claim 11, wherein the direct current is within a range of 0-500V.

13. The hand-held gemstone testing apparatus of claim 9, wherein the current transmitting electrode comprises a contact touch pad.

14. The hand-held gemstone testing apparatus of claim 9, wherein the current transmitting electrode is embodied within a handgrip disposed at the body proximal end.

15. The hand-held gemstone testing apparatus of claim 9, wherein the electric probe is integrated within the test probe.

16. The hand-held gemstone testing apparatus of claim 9, wherein the test probe is configured as both the current transmitting electrode and the tubular optical transmission channel.

17. The hand-held gemstone testing apparatus of claim 9, the signals sensed by the electric probe comprises a conductivity measurement, and the electronic testing system is configured to generate the gemstone test result as a function of an UV absorbance of the gemstone test specimen and the conductivity measurement.

18. The hand-held gemstone testing apparatus of claim 9, wherein the handheld body comprises an elongated body.

19. A gemstone testing apparatus operating method comprising:
in response to a switch signal generated by engaging a gemstone specimen at a proximal end of a gemstone testing device, activate a plurality of light sources configured to UV light rays in a proximal directly away from the gemstone testing device to illuminate a gemstone specimen;
activate a stimulus electrode configured to provide a direct electric current through a touch pad to the gemstone specimen;
receive an intensity measurement signal of the UV light rays reflected from the gemstone specimen through a test probe comprising a tubular optical transmission channel;
receive an electric current measurement signal of the electric current conducted through the gemstone specimen from the test probe;
determine a gemstone test result in accordance to signals received from the test probe; and
transmit an electrical signal regarding the determined gemstone test result to a buzzer, such that a moissanite of a colorless range is to be detected.

20. The gemstone testing apparatus operating method of claim 19, further comprises:
receive an electric test disable signal, wherein the stimulus electrode is deactivated upon receiving the electric test disable signal, and the gemstone test result is determined based solely on the intensity measurement signal.
